(19) United States
(12) Patent Application Publication
(10) Pub. No.: US 2020/0205664 A1
(43) Pub. Date: Jul. 2, 2020

(54) METHOD AND SYSTEM FOR MULTI-MODAL IMAGING OF TISSUE

(71) Applicant: Agency for Science, Technology and Research, Singapore (SG)

(72) Inventors: Jun Hui Chris Ho, Singapore (SG); Renzhe Bi, Singapore (SG); Chi Lok Dave Wong, Singapore (SG); Malini Olivo, Singapore (SG); Tao Yang, Singapore (SG); Zhongkang Lu, Singapore (SG); Weimin Huang, Singapore (SG); Kapil Dev, Singapore (SG)

(21) Appl. No.: 16/622,038

(22) PCT Filed: Jul. 3, 2018
(86) PCT No.: PCT/SG2018/050325

§ 371 Date: Dec. 12, 2019

(30) Foreign Application Priority Data
Jul. 3, 2017 (SG) 10201705474V

(51) Int. Cl. A61B 5/00; A61B 5/055

(52) U.S. Cl. CPC A61B 5/0035; A61B 2090/306; A61B 5/055; A61B 5/0059

(57) ABSTRACT

There is provided a method for multi-modal imaging of tissue. The method includes: providing a fiber probe over a target area of the tissue, the fiber probe including a plurality of source fiber channels, a plurality of detector fiber channels and a plurality of fiducial marker channels, the plurality of source fiber channels, the plurality of detector fiber channels and the plurality of fiducial marker channels having a predetermined spatial arrangement in the fiber probe; directing light through each of the plurality of source fiber channels of the fiber probe to the target area in succession for performing optical imaging of the target area; obtaining optical imaging data of the target area based on attenuated light from the target area detected through the plurality of detector fiber channels due to said light directed to the target area; obtaining MRI data of the target area based on a MRI of the target area; and coregistering the optical imaging data and the MRI data based on a plurality of fiducial features captured in the MRI data corresponding to the plurality of fiducial marker channels of the fiber probe. There is also provide a corresponding system for multi-modal imaging of tissue.

### CROSS-REFERENCE TO RELATED APPLICATION

This application claims the benefit of priority of Singapore Patent Application No. 10201705474V filed 3 Jul. 2017, the content of which being hereby incorporated by reference in its entirety for all purposes.

### TECHNICAL FIELD

The present invention generally relates to a method for multi-modal imaging of tissue, and a system thereof, and in particular, for combining magnetic resonance imaging (MRI) and optical imaging (e.g., diffuse optical imaging).

### BACKGROUND

Diffuse optical tomography (DOT) is an optical imaging technique, which can be used to measure tissue oxygenation and blood perfusion using diffuse light. In the DOT technique, for example, coherent light is shone into tissue through a source fiber. The light is absorbed and scattered within the tissue, and attenuated light is collected a certain distance away through a detector fiber. In this manner, the amount of light absorbed and scattered in the banana-shaped sensitivity volume is indirectly measured through the amount of light attenuation at the detector end.

In the near-infrared (NIR) wavelength range (700-900 nm), the low absorption of light by tissue can be exploited for deep tissue imaging. The main tissue absorbers in this near-infrared window are oxy- and deoxy-hemoglobin, and melanin. In this regard, since light absorption is a function of extinction coefficient and concentration of tissue absorbers in tissue, if the extinction coefficients of tissue absorbers as a function of wavelength are known and light absorption in tissue can be measured, then the concentration of these tissue absorbers can be computed. Using the concentration of oxy- and deoxy-hemoglobin, the oxygen saturation and total hemoglobin concentration in tissue can be calculated, which can provide physiological information about tissue vasculature. In addition, the intensity fluctuation of the attenuated light detected can also indicate the blood perfusion within tissue, of which an optical imaging technique known as diffuse speckle contrast analysis (DSCA) is based on. Moreover, the combination of oxy/deoxy-hemoglobin concentration and blood perfusion can provide the information on the metabolism rate of the targeted tissue, which is directly affected by many diseases, such as stroke, brain obstruction, coronary heart disease and lower limb ulcers.

On the other hand, magnetic resonance imaging (MRI) may provide complementary information and improve the characterization of the targeted tissue. For example, DOT may be used to obtain the chromophore concentration maps, such as oxy- and deoxy-hemoglobin, water, and fat, whereas MRI provides high-resolution structural and functional information, such as water, fat and vascular volume. MRI may also provide water and fat concentrations and vascular volume information that can be used to improve the reconstruction accuracy of the oxy- and deoxy-hemoglobin, and thus the detection and characterization of various diseases, such as tumor, due to the associated vasculature.

Therefore, it may be desirable to provide a multi-modal imaging method, including optical imaging and MRI. However, there exist various problems associated with conventional multi-modal imaging methods. For example, conventional multi-modal imaging methods may suffer from limited spatial resolution and tissue penetration depth, difficulties in MRI/optical spatial image registration, scalability issues or difficulties relating to multimodal MRI/optical imaging to MRI coils of different sizes for various MRI preclinical/clinical imaging applications, and the lack of DSCA incorporation/integration.

A need therefore exists to provide a method of multi-modal imaging of tissue, and a system thereof, that seek to overcome, or at least ameliorate, one or more of the deficiencies of conventional multi-modal imaging methods, and in particular, for combining MRI and optical imaging (e.g., diffuse optical imaging). It is against this background that the present invention has been developed.

### SUMMARY

According to a first aspect of the present invention, there is provided a method for multi-modal imaging of tissue, the method comprising:

providing a fiber probe over a target area of the tissue, the fiber probe comprising a plurality of source fiber channels, a plurality of detector fiber channels and a plurality of fiducial marker channels, the plurality of source fiber channels, the plurality of detector fiber channels and the plurality of fiducial marker channels having a predetermined spatial arrangement in the fiber probe;

directing light through each of the plurality of source fiber channels of the fiber probe to the target area in succession for performing optical imaging of the target area;

obtaining optical imaging data of the target area based on attenuated light from the target area detected through the plurality of detector fiber channels due to said light directed to the target area;

obtaining magnetic resonance imaging (MRI) data of the target area based on a MRI of the target area; and

coregistering the optical imaging data and the MRI data based on a plurality of fiducial features captured in the MRI data corresponding to the plurality of fiducial marker channels of the fiber probe.

According to a second aspect of the present invention, there is provided a system for multi-modal imaging of tissue, the system comprising:
an optical imaging system comprising:
   a fiber probe configured to be provided over a target area of the tissue during the multi-model imaging of the target area, the fiber probe comprising a plurality of source fiber channels, a plurality of detector fiber channels and a plurality of fiducial marker channels, the plurality of source fiber channels, the plurality of detector fiber channels and the plurality of fiducial marker channels having a predetermined spatial arrangement in the fiber probe;
   an optical switch configured to direct light through each of the plurality of source fiber channels of the fiber probe to the target area in succession for performing optical imaging of the target area; and
   an image capturing module configured to obtain optical imaging data of the target area based on attenuated light from the target area detected through the plurality of detector fiber channels due to said light directed to the target area;
a magnetic resonance imaging (MRI) system comprising:
   an image capturing module configured to obtain MRI data of the target area based on a MRI of the target area; and
an image coregistration module configured to coregister the optical imaging data and the MRI data based on a plurality of fiducial features captured in the MRI data corresponding to the plurality of fiducial marker channels of the fiber probe.

### BRIEF DESCRIPTION OF THE DRAWINGS

Embodiments of the present invention will be better understood and readily apparent to one of ordinary skill in the art from the following written description, by way of example only, and in conjunction with the drawings, in which:

FIG. 1 depicts a schematic flow diagram of a method for multi-modal imaging of tissue according to various embodiments of the present invention;

FIG. 2A depicts a schematic drawing of an example system configured for DCS;

FIG. 2B depicts a schematic drawing of an example system configured for DSCA;

FIG. 3 depicts a schematic drawing of a system for multi-modal imaging of tissue according to various embodiments of the present invention;

FIG. 4 depicts a schematic drawing of a fiber probe according to various embodiments of the present invention;

FIG. 5A depicts a schematic drawing of a fiber probe (multi-modal MRI-optical probe with customized source-detector and fiducial channel arrangements) according to various example embodiments of the present invention;

FIG. 5B depicts an image of an exemplary implementation of an exemplary probe coupled with an MRI surface coil for use in an example multi-modal MRI-DOT imaging according to various example embodiments of the present invention;

FIG. 6 depicts a schematic drawing of an exemplary system for multi-modal imaging of tissue according to various example embodiments of the present invention;

FIG. 7 depicts a flow chart showing various functions of the control system (PC for data acquisition) shown in FIG. 6;

FIGS. 8A and 8B depict exemplary graphical user interfaces (GUIs) associated with various functions of the control system shown in FIG. 6;

FIG. 9 depicts a raw CCD image (raw optical image) corresponding to a particular source channel according to an example embodiment of the present invention;

FIG. 10A depicts a MRI anatomical image of a mouse brain whereby the glycerin-filled fiducial channels on the probe show up as high intensity regions;

FIG. 10B depicts a MRI anatomical image with the MRI-invisible source channels and detector channels locations derived from the glycerin-filled fiducial channels based on prior knowledge of the specific arrangement overlaid on the MRI anatomical image;

FIG. 11 depicts a timeline of the imaging cycles of the NIRS and MRI systems according to various example embodiment of the present invention;

FIG. 12 depicts a flow diagram showing an overview of the method of multi-modal imaging of tissue according to various example embodiments of the present invention;

FIG. 13 depicts 3D absorption map of India ink-Intralipid mixture flowing in a phantom channel, in 3 orthogonal planes and 3D-rendered visualization according to an example embodiment of the present invention;

FIG. 14 depicts 3D flow map of India ink-Intralipid mixture flowing in a phantom channel, in 3 orthogonal planes and 3D-rendered visualization according to an example embodiment of the present invention; and

FIGS. 15A and 15B depict images relating to overlaid optical data from NIRS (oxygen saturation) and MRI image stack.

### DETAILED DESCRIPTION

Various embodiments of the present invention provide a method for multi-modal imaging of tissue, and a system thereof, and in particular, for combining magnetic resonance imaging (MRI) and optical imaging (e.g., diffuse optical imaging).

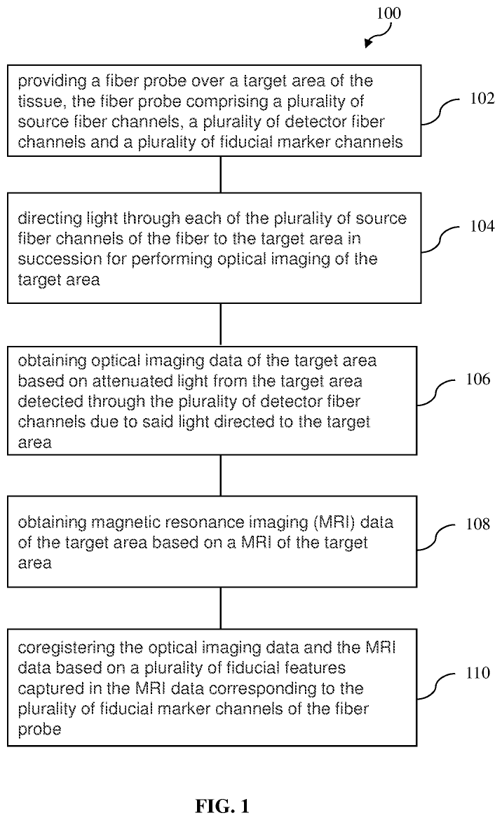

FIG. 1 depicts a schematic flow diagram of a method 100 for multi-modal imaging of tissue according to various embodiments of the present invention. The method 100 comprises a step 102 of providing a fiber probe over a target area of the tissue. In particular, the fiber probe comprises a plurality of source fiber channels, a plurality of detector fiber channels and a plurality of fiducial marker channels. Furthermore, the plurality of source fiber channels, the plurality of detector fiber channels and the plurality of fiducial marker channels have a predetermined spatial arrangement in the fiber probe. The method 100 further comprises a step 104 of directing light through each of the plurality of source fiber channels of the fiber probe to the target area in succession (i.e., one after another) for performing optical imaging of the target area; a step 106 of obtaining optical imaging data of the target area based on attenuated light from the target area detected through the plurality of detector fiber channels due to the light directed to the target area; a step 108 of obtaining magnetic resonance imaging (MRI) data of the target area based on a MRI of the target area; and a step 110 of coregistering the optical imaging data and the MRI data based on a plurality of fiducial features captured in the MRI data corresponding to the plurality of fiducial marker channels of the fiber probe.

In relation to step 102, for example, the fiber probe may be moved to a position/location over a target area of interest of the tissue (i.e., organism tissue, such as human or animal body tissue) desired for optical imaging. In various embodiments, the optical imaging is based on a diffuse optical imaging technique, such as but not limited to, diffuse optical tomographic (DOT), diffuse correlation spectroscopy (DCS) and/or diffuse speckle contrast analysis (DSCA). For example, as explained in the background, DOT is an optical imaging technique, which can be used to measure tissue oxygenation and blood perfusion using diffuse light. In the DOT technique, for example, coherent light is shone into tissue through a source fiber (which also may be referred to as an emitter). The light is absorbed and scattered within the tissue, and attenuated light is collected a certain distance away through a detector fiber (or simply referred to as a detector). In this manner, the amount of light absorbed and scattered in a banana-shaped sensitivity volume is indirectly measured through the amount of light attenuation at the detector end.

As also explained in the background, in the near-infrared (NIR) wavelength range (700-900 nm), the low absorption of light by tissue can be exploited for deep tissue imaging. The main tissue absorbers in this near-infrared window are oxy- and deoxy-hemoglobin, and melanin. In this regard, since light absorption (μ_{a,total}) is a function of extinction coefficient (or absorption coefficient) (μₐ) and concentration of tissue absorbers (c) in tissue, that is, μ_{a,total}=ƒ(μₐ,c), if the extinction coefficients of tissue absorbers as a function of wavelength are known and light absorption in tissue can be measured, then the concentration of these tissue absorbers can be computed. Using the concentration of oxy- and deoxy-hemoglobin, the oxygen saturation and total hemoglobin concentration in tissue can be calculated, which can provide physiological information about tissue vasculature. In addition, the intensity fluctuation of the attenuated light detected can also indicate the blood perfusion within tissue, of which an optical imaging technique known as DSCA is based on. Moreover, the combination of oxy/deoxy-hemoglobin concentration and blood perfusion can provide the information on the metabolism rate of the targeted tissue, which is directly affected by many diseases, such as stroke, brain obstruction, coronary heart disease and lower limb ulcers.

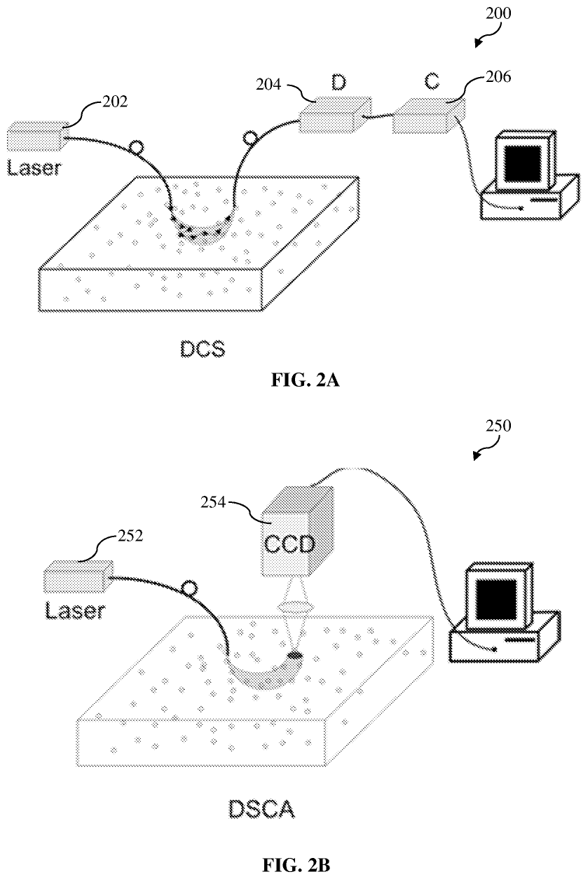

For illustration purpose only and without limitation, FIG. 2A depicts a schematic drawing of an example system 200 configured for DCS and FIG. 2B depicts a schematic drawing of an example system 250 configured for DSCA. For example, for deep tissue blood perfusion measurement, there are two optical modalities, DCS and DSCA. As shown in FIG. 2A, the DCS system 200 may use a coherent laser 202 as a light source and a high sensitivity photon detector 204 together with a correlator 206 as a detector. The DCS system 200 may require a data fitting algorithm/technique to extract perfusion information. As shown in FIG. 2B, the DSCA system 250 may use a coherent laser 252 as a light source and an industrial CCD camera 254 as a detector. The DSCA system 250 uses laser speckle contrast to indicate blood perfusion.

In relation to steps 104 and 106, in various embodiments, the optical imaging data comprises at least one set of optical images (i.e., images obtained optical imaging), each set comprising a plurality of optical images. In this regard, each optical image in a set is generated based on the attenuated light detected through the plurality of detector fiber channels due to the light directed through a respective one of the plurality of source fiber channels.

In various embodiments, each optical image comprises a plurality of light spots, each of the plurality of light spots corresponding to the attenuated light detected from a respective one of the plurality of detector fiber channels due to the light directed through the source fiber channel associated with the optical image. In this regard, the source fiber channel forms a source-detector pair with each respective one of the plurality of detector fiber channels, and each of the plurality of light spots is generated based on a respective one of the plurality of source-detector pairs.

For example, when light is directed through a particular/selected one of the plurality of source fiber channels to the target area, attenuated light from the target area may be detected through each of the plurality of detector fiber channels, resulting in an optical image comprising a plurality of light spots (corresponding to the attenuated light detected from the plurality of detector fiber channels). Accordingly, by directing light through each of the plurality of source fiber channels in succession (i.e., one after another), a set of optical images may be generated. In various embodiments, such a step of directing light through each of the plurality of source fiber channels in succession may be repeated by a certain or predetermined number of times (or rounds), thereby resulting in a plurality of sets of optical images, each set of optical images generated by a corresponding round of optical imaging. For example, directing light through a source fiber channel in succession has been advantageously found to improve the quality of the corresponding optical images obtained for each source fiber channel. For example, in such a manner, the resulting CCD image acquired for a particular source fiber channel contains information on the attenuated light intensity for all the source-detector pairs corresponding to the particular source fiber channel, with each bright spot on the CCD image corresponding to the attenuated light from a respective detector fiber channel when light impinges onto the sample via the particular source fiber channel.

Accordingly, each light spot in the optical image may be considered as a result of a corresponding source-detector pair, that is, generated based on the attenuated light detected from the corresponding detector fiber channel due to the original light directed to the target area through the corresponding source fiber channel.

In various embodiments, the plurality of source-detector pairs is arranged in the fiber probe so as to have a range of source-detector distances. In various embodiments, the range of source-detector distances may be from about 1 mm to about 60 mm. It will be appreciated by a person skilled in the art that the range of source-detector distances may be configured as desired or as appropriate based on various factors, such as the power of the incident light in the source fiber channel (e.g., if the source-detector distance becomes too large, the attenuated light may become undesirably weak), the desired spatial resolution, the desired tissue penetration depth, and so on. For example, the plurality of source-detector pairs (source-detector arrangement) may be configured as appropriate to achieve a desired spatial resolution and/or tissue penetration depth for optical imaging. For example, different permutations of source-detector pair distances may be configured as desired or as appropriate to probe different depths of the tissue.

In various embodiments, each of the fiducial marker channels has disposed therein a material that is capable of being captured by MRI (MRI-visible). For example, the fiducial marker channels may each be filled with such a material. Therefore, the positions of the fiducial marker channels in the fiber probe may be identified or determined in the MRI images for facilitating the process of coregistering the optical imaging data and the MRI data based on the plurality of fiducial features (corresponding to the plurality of fiducial marker channels) captured in the MRI data. In various embodiments, the material is, but not limited to, glycerin. It will be appreciated by a person skilled in the art that other types of material may be used as desired or as appropriate, as long as the material is MRI-visible and sufficiently tolerant to MRI.

In various embodiments, the MRI and the optical imaging of the target area are performed simultaneously. Furthermore, step 110 of coregistering the optical imaging data and the MRI data comprises temporal coregistering and spatial coregistering the optical imaging data and the MRI data.

In various embodiments, the method 100 further comprises, for an imaging cycle (or for each imaging cycle), sending a trigger signal from a MRI system configured to perform the MRI to an optical imaging system configured to perform the optical imaging to start the optical imaging for facilitating the above-mentioned temporal coregistering the optical imaging data and the MRI data. For example, the MRI and the optical imaging may be advantageously synchronized, thus enabling the MRI data and the optical imaging data to be temporally aligned.

In various embodiments, the above-mentioned spatial coregistering comprises spatially aligning a MRI image (or each MRI image) of the MRI data and an optical image (or each optical imaging image) of the optical imaging data based on positions of the plurality of fiducial features captured in the MRI image and positions of the plurality of light spots captured in the optical image, and based on the predetermined spatial arrangement of the plurality of detector fiber channels and the plurality of fiducial marker channels in the fiber probe. For example, since the plurality of source fiber channels, the plurality of detector fiber channels and the plurality of fiducial marker channels in the fiber probe have a predetermined spatial arrangement (thus, their relative position to each other is known), the MRI image (having the positions of the plurality of fiducial features captured therein) and the optical image (having the positions of the plurality of light spots captured therein (corresponding to the plurality of detector fiber channels)) may be spatially aligned with each other based on the known positions of the plurality of fiducial marker channels relative to the positions of the plurality of detector fiber channels, or vice versa.

Accordingly, various embodiments of the present invention advantageously provide a method for multi-modal imaging of tissue, in particular, for combining optical imaging and MRI, that is effective and efficient. For example, the fiber probe is advantageously configured to include a plurality of source fiber channels and a plurality of detector fiber channels (thus, a plurality of source-detector pairs) for optical imaging (e.g., diffuse optical imaging) a target area, and integrated with a plurality of fiducial marker channels that is MRI-visible such that the positions of the fiducial marker channels can be captured in the MRI image, which may then be used for coregistering the MRI data with the optical imaging data. Furthermore, the source-detector pairs may be arranged in fiber probe as appropriate to achieve a desired spatial resolution and/or tissue penetration depth. As a result, optical imaging data and MRI data may advantageously be obtained simultaneously and be coregistered.

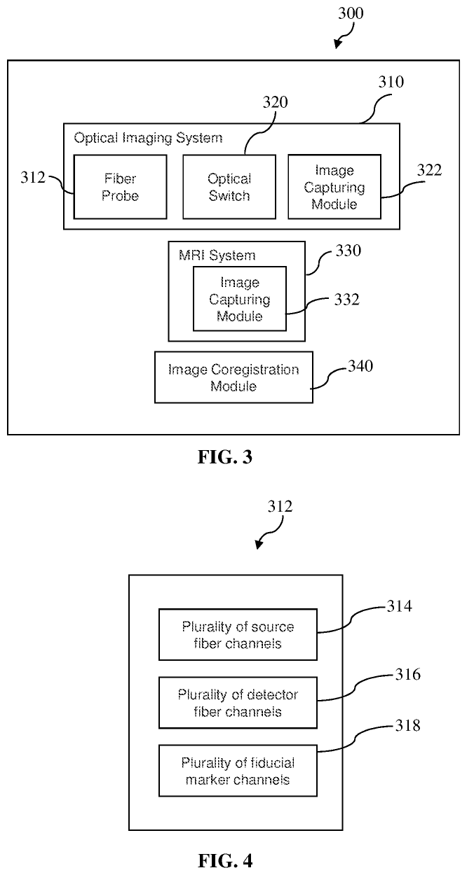

FIG. 3 depicts a schematic drawing of a system 300 for multi-modal imaging of tissue (or may be referred to as a multi-modal imaging system) according to various embodiments of the present invention, such as corresponding to the method 100 for multi-modal imaging of tissue as described hereinbefore according to various embodiments of the present invention. The system 300 comprises an optical imaging system 310 (e.g., diffuse optical imaging system, such as a DOT imaging system, or NIR spectroscopy system) including: a fiber probe 312 configured to be provided over a target area of the tissue for performing multi-modal imaging of the target area, the fiber probe 312 comprising a plurality of source fiber channels 314, a plurality of detector fiber channels 316 and a plurality of fiducial marker channels 318, the plurality of source fiber channels 314, the plurality of detector fiber channels 316 and the plurality of fiducial marker channels 318 having a predetermined spatial arrangement in the fiber probe 312.

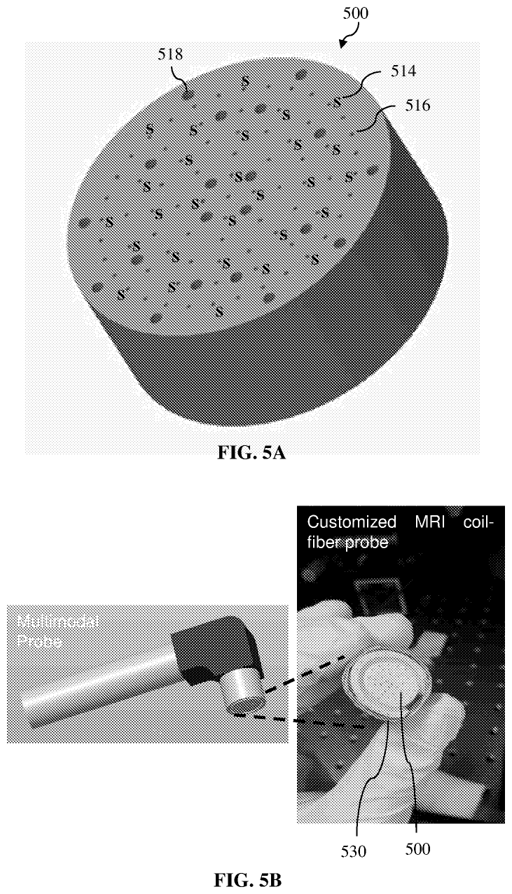

FIG. 4 depicts a schematic drawing of the fiber probe 312 according to various embodiments of the present invention. It can be understood by a person skilled in the art that FIG. 4 does not illustrate any specific configuration or form of the fiber probe 312, but merely show the existence of various components of the fiber probe 312.

The optical imaging system 310 further comprises an optical switch 320 configured to direct light through each of the plurality of source fiber channels 314 of the fiber probe 312 to the target area in succession for performing optical imaging of the target area; and an image capturing module 322 configured to obtain optical imaging data of the target area based on attenuated light from the target area detected through the plurality of detector fiber channels 316 due to the light directed to the target area.

The system 300 further comprises a MRI system 330 including an image capturing module 332 configured to obtain MRI data of the target area based on a MRI of the target area, and an image coregistration module 340 configured to coregister the optical imaging data and the MRI data based on a plurality of fiducial features captured in the MRI data corresponding to the plurality of fiducial marker channels 318 of the fiber probe 312.

It will be appreciated by a person skilled in the art that each of the optical imaging system 310 and MRI system 330 may comprise a memory and at least one processor communicatively coupled to the memory and configured to perform various functions/operations as described hereinbefore according to various embodiments. For example, with respect to the optical imaging system 310, at least one processor may be configured to control the optical switch 320 to direct light through each of the plurality of source fiber channels 314 of the fiber probe 312 to the target area in succession for performing optical imaging of the target area; and control the image capturing module 322 to obtain optical imaging data of the target area based on attenuated light from the target area detected through the plurality of detector fiber channels 316 due to the light directed to the target area. Similarly, with respect to the MRI system 330, at least one processor may be configured to control the image capturing module 332 to obtain MRI data of the target area based on a MRI of the target area.

It will also be appreciated by a person skilled in the art that at least one processor may be configured to perform the required functions or operations through set(s) of instructions (e.g., software modules) executable by the at least one processor to perform the required functions or operations, such as to realize the image coregistration module 340.

In various embodiments, the system 300 corresponds to the method 100 as described hereinbefore with reference to FIG. 1, and therefore, various functions or operations configured to be performed by system 300 may correspond to various steps of the method 100 described hereinbefore according to various embodiments, and thus need not be repeated with respect to the system 300 for clarity and conciseness. In other words, various embodiments described herein in context of the methods are analogously valid for the respective systems or devices, and vice versa.

A computing system, a controller, a microcontroller or any other system providing a processing capability may be provided according to various embodiments in the present disclosure. Such a system may be taken to include one or more processors and one or more computer-readable storage mediums. For example, the system 300 described hereinbefore may include a processor (or controller) and a computer-readable storage medium (or memory) which are for example used in various processing carried out therein as described herein. A memory or computer-readable storage medium used in various embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In various embodiments, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g., a microprocessor (e.g., a Complex Instruction Set Computer (CISC) processor or a Reduced Instruction Set Computer (RISC) processor). A “circuit” may also be a processor executing software, e.g., any kind of computer program, e.g., a computer program using a virtual machine code, e.g., Java. Any other kind of implementation of the respective functions which will be described in more detail below may also be understood as a “circuit” in accordance with various alternative embodiments. Similarly, a “module” may be a portion of a system according to various embodiments in the present invention and may encompass a “circuit” as above, or may be understood to be any kind of a logic-implementing entity therefrom.

Some portions of the present disclosure are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

In addition, the present specification also at least implicitly discloses a computer program or software/functional module, in that it would be apparent to the person skilled in the art that various steps of the methods described herein (e.g., step 110 of coregistering the optical imaging data and the MRI data) may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention. It will be appreciated by a person skilled in the art that various modules described herein (e.g., image coregistration module 340) may be software module(s) realized by computer program(s) or set(s) of instructions executable by a computer processor to perform the required functions, or may be hardware module(s) being functional hardware unit(s) designed to perform the required functions. It will also be appreciated that a combination of hardware and software modules may be implemented.

Furthermore, one or more of the steps of a computer program/module or method described herein may be performed in parallel rather than sequentially.

It will be appreciated by a person skilled in the art that the terminology used herein is for the purpose of describing various embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

In order that the present invention may be readily understood and put into practical effect, various example embodiments of the present invention will be described hereinafter by way of examples only and not limitations. It will be appreciated by a person skilled in the art that the present invention may, however, be embodied in various different forms or configurations and should not be construed as limited to the example embodiments set forth hereinafter. Rather, these example embodiments are provided so that this disclosure will be thorough and complete, and will fully convey the scope of the present invention to those skilled in the art.

Various example embodiments of the present invention provides a high-resolution high-sensitivity multi-modal MRI-DOT imaging system.

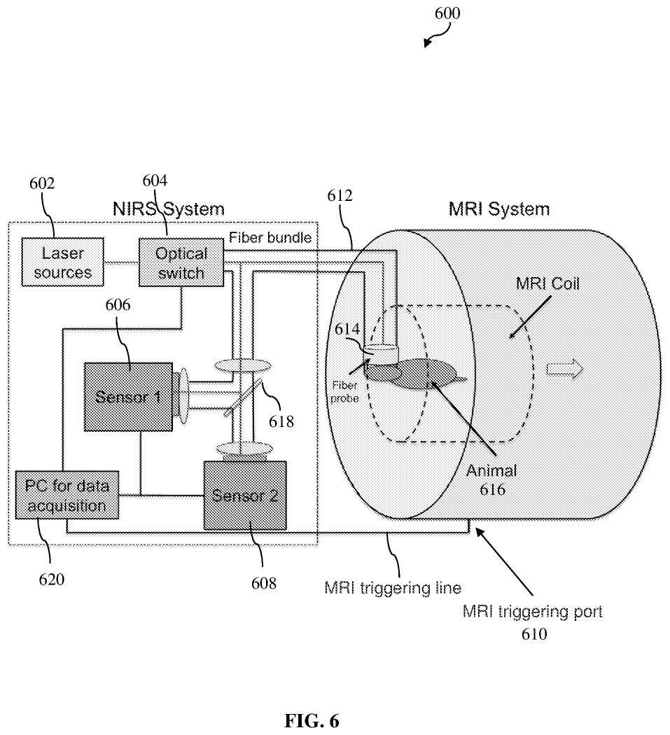

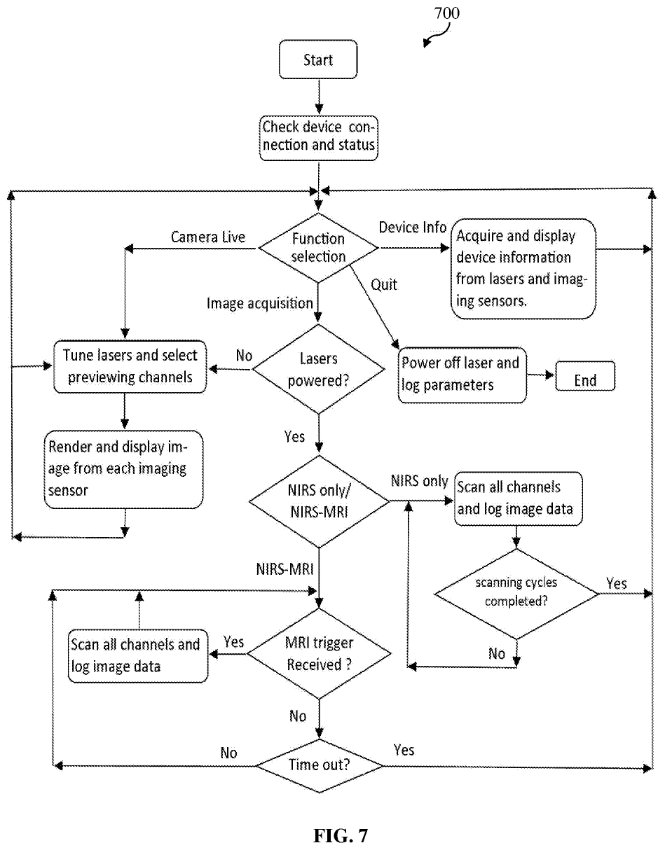

According to various example embodiments, an exemplary multi-modal MRI-DOT optical probe 500 is provided as shown in FIG. 5A, which can be used to concurrently measure multiple physiological parameters in tissues, such as oxygenation and perfusion, using both MRI and optical imaging modalities. The probe 500 is configured to couple a high-density optical probe arrangement with a high-sensitivity MRI surface coil for high-resolution high-sensitivity multimodal imaging. Coupled with an exemplary multi-modal MRI-DOT system as shown in FIG. 6 according to various example embodiments, the probe 500 can be used to perform optical measurements, which can be exploited to measure tissue oxygenation and perfusion simultaneously using DOT and DSCA, which can be validated by blood oxygenation-level dependent (BOLD) and blood perfusion measurements from MRI, respectively.

In the probe illustrated in FIG. 5A, the fiducial marker channels 518 correspond to the larger channels, and the source fiber channels 514 and the detector fiber channels 516 correspond to the smaller channels. To differentiate the source fiber channels 514 and the detector fiber channels 516 in FIG. 5A, each source fiber channel 514 is denoted by ‘S’ (and thus each smaller channel not denoted by ‘S’ corresponds to a detector fiber channel). The source-detector arrangement and geometry on the probe 500 can be optimized for a desired resolution-penetration depth configuration for multiple imaging applications. Moreover, MRI-visible fiducial marker channels 518 may be distributed uniformly throughout the probe 500 for accurate MRI-optical image registration. It will be appreciated by a person skilled in the art that the source fiber channels 514, detector fiber channels 516 and fiducial marker channels 518 are not limited to the configuration (e.g., number and spatial arrangement) as shown in FIG. 5A, which is shown for illustration purpose only and without limitation. In particular, it will be appreciated by a person skilled in the art that various configurations/arrangements may be implemented for various purposes, such as based on the desired spatial resolution and/or tissue penetration depth. It will also be appreciated by a person skilled in the art that the probe is not limited to having a circular cross-section and may be realized in various other shapes as desired or as appropriate, such as rectangular or square. In the probe 500, as shown, each source fiber channel 514, each detector fiber channel 516 and each fiducial marker channel 518 extend completely through the length of the probe 500 so as to form corresponding openings on both sides (e.g., top and bottom sides) of the probe 500.

By way of example only and without limitation, the probe 500 may have a length of 100 μm and a diameter of 19 mm. In addition, there may be provided 28 source fiber channels 514, 49 detector fiber channels 516 and 20 fiducial marker channels 518. Furthermore, each source fiber channel 514, each detector fiber channel 516 and each fiducial marker channel 518 may be arranged in the probe 500 at the following positions/coordinates (x, y, z) with respect to a center of the probe 500:

TABLE 1
Positions/coordinates (x, y, z) of the fiducial marker channels

| Number | x | y | z |
|---|---|---|---|
| 1 | 3.33 | 7.79 | 0 |
| 2 | 8.5 | 0 | 0 |
| 3 | 7.16 | −4.78 | 0 |
| 4 | 3.33 | −7.79 | 0 |
| 5 | −3.33 | −7.79 | 0 |
| 6 | −7.16 | −4.78 | 0 |
| 7 | −8.5 | 0 | 0 |
| 8 | −3.33 | 7.79 | 0 |
| 9 | 1.17 | 5.88 | 0 |
| 10 | 4.99 | 3.33 | 0 |
| 11 | 4.99 | −3.33 | 0 |
| 12 | 1.17 | −5.88 | 0 |
| 13 | −1.17 | −5.88 | 0 |
| 14 | −4.99 | −3.33 | 0 |
| 15 | −4.99 | 3.33 | 0 |
| 16 | −1.17 | 5.88 | 0 |
| 17 | 1.17 | 1.17 | 0 |
| 18 | 1.17 | −1.17 | 0 |
| 19 | −1.17 | −1.17 | 0 |
| 20 | −1.17 | 1.17 | 0 |

TABLE 2
Positions/coordinates (x, y, z) of the source fiber channels

| Number | x | y | z |
|---|---|---|---|
| 1 | 0 | 7.5 | 0 |
| 2 | 5.3 | 5.3 | 0 |
| 3 | 7.5 | 0 | 0 |
| 4 | 5.3 | −5.3 | 0 |
| 5 | 0 | −7.5 | 0 |
| 6 | −5.3 | −5.3 | 0 |
| 7 | −7.5 | 0 | 0 |
| 8 | −5.3 | 5.3 | 0 |
| 9 | −2.3 | 5.54 | 0 |
| 10 | 2.3 | 5.54 | 0 |
| 11 | 5.54 | 2.3 | 0 |
| 12 | 5.54 | −2.3 | 0 |
| 13 | 2.3 | −5.54 | 0 |
| 14 | −2.3 | −5.54 | 0 |
| 15 | −5.54 | −2.3 | 0 |
| 16 | −5.54 | 2.3 | 0 |
| 17 | −3.18 | 3.18 | 0 |
| 18 | 0 | 4.5 | 0 |
| 19 | 3.18 | 3.18 | 0 |
| 20 | 4.5 | 0 | 0 |
| 21 | 3.18 | −3.18 | 0 |
| 22 | 0 | −4.5 | 0 |
| 23 | −3.18 | −3.18 | 0 |
| 24 | −4.5 | 0 | 0 |
| 25 | 0 | 1.5 | 0 |
| 26 | 1.5 | 0 | 0 |
| 27 | 0 | −1.5 | 0 |
| 28 | −1.5 | 0 | 0 |

TABLE 3
Positions/coordinates (x, y, z) of the detector fiber channels

| Number | x | y | z |
|---|---|---|---|
| 1 | 1.46 | 7.36 | 0 |
| 2 | 2.87 | 6.93 | 0 |
| 3 | 4.17 | 6.24 | 0 |
| 4 | 6.24 | 4.17 | 0 |
| 5 | 6.93 | 2.87 | 0 |
| 6 | 7.36 | 1.46 | 0 |
| 7 | 7.36 | −1.46 | 0 |
| 8 | 6.93 | −2.87 | 0 |
| 9 | 6.24 | −4.17 | 0 |
| 10 | 4.17 | −6.24 | 0 |
| 11 | 2.87 | −6.93 | 0 |
| 12 | 1.46 | −7.36 | 0 |
| 13 | −1.46 | −7.36 | 0 |
| 14 | −2.83 | −6.93 | 0 |
| 15 | −4.17 | −6.24 | 0 |
| 16 | −6.24 | −4.17 | 0 |
| 17 | −6.93 | −2.87 | 0 |
| 18 | −7.36 | −1.46 | 0 |
| 19 | −7.36 | 1.46 | 0 |
| 20 | −6.93 | 2.87 | 0 |
| 21 | −6.24 | 4.17 | 0 |
| 22 | −4.17 | 6.24 | 0 |
| 23 | −2.83 | 6.93 | 0 |
| 24 | −1.46 | 7.36 | 0 |
| 25 | 0 | 6 | 0 |
| 26 | 4.24 | 4.24 | 0 |
| 27 | 6 | 0 | 0 |
| 28 | 4.24 | −4.24 | 0 |
| 29 | 0 | −6 | 0 |
| 30 | −4.24 | −4.24 | 0 |
| 31 | −6 | 0 | 0 |
| 32 | −4.24 | 4.24 | 0 |
| 33 | −1.72 | 4.16 | 0 |
| 34 | 1.72 | 4.16 | 0 |
| 35 | 4.16 | 1.72 | 0 |
| 36 | 4.16 | −1.72 | 0 |
| 37 | 1.72 | −4.16 | 0 |
| 38 | −1.72 | −4.16 | 0 |
| 39 | −4.16 | −1.72 | 0 |
| 40 | −4.16 | 1.72 | 0 |
| 41 | −2.12 | 2.12 | 0 |
| 42 | 0 | 3 | 0 |
| 43 | 2.12 | 2.12 | 0 |
| 44 | 3 | 0 | 0 |
| 45 | 2.12 | −2.12 | 0 |
| 46 | 0 | −3 | 0 |
| 47 | −2.12 | −2.12 | 0 |
| 48 | −3 | 0 | 0 |
| 49 | 0 | 0 | 0 |

In various example embodiments, the probe 500 may be made of plastic for MRI compatibility, and the source-detector arrangement may be specially designed to simultaneously achieve sufficient spatial resolution and tissue penetration depth for the region-of-interest it is probing. The source channels 514 and detector channels 516 may be configured to have a dense packing arrangement to achieve high spatial resolution, and different permutations of source-detector pairs may be realized for probing different tissue depths.

In various example embodiments, the probe surface area is configured to at least cover the region of interest laterally. For example, the tissue penetration depth may be roughly half the largest source-detector distance (between the source channel 514 and detector channel 516 farthest from each other), which should be sufficient to also cover the region of interest depth-wise. In various example embodiments, the minimum spacing between a source channel 514 and a detector channel 516 is about 1 mm to ensure that the photon propagation in tissue is in the diffuse regime, as a model of diffuse light propagation in tissue is used in the downstream data processing. The source channels 514 and detector channels 516 may be configured to have a circular arrangement as shown in FIG. 5A, which may be preferred to image organs/tumors approximately spherical in geometry, but is not limited to such a circular arrangement. For example, a rectangular arrangement may be formed to image organs which are more elongated in geometry.

FIG. 5B depicts an image of an exemplary implementation of the probe 500 coupled with an MRI surface coil 530 for use in multi-modal MRI-DOT imaging for illustration purpose only and without limitation.

FIG. 6 depicts a schematic drawing of an exemplary system 600 for multi-modal imaging of tissue according to various example embodiments of the present invention. The system 600 comprises laser sources 602, an optical multiplexer (or optical switch) 604, optical imaging sensors (e.g., a first image sensor 606 and a second image sensor 608), a triggering port 610 connected to the MRI system, long optical fibers 612 attached to the optical tomographic probe 614 (e.g., corresponding to the fiber probe as described hereinbefore according to various embodiments of the present invention), as well as other optical components. For example, multiple laser sources with different wavelengths may be incorporated in the system 600 via a laser beam combiner into a single optical path. The optical multiplexer 604 directs the optical path of the light into a specific source channel on the probe 614. The attenuated light from the sample 616 is collected by detector fibers attached to the probe 614 and then separated into two optical paths according to wavelength via a beam splitter 618, each then projected onto its respective optical imaging sensor 606, 608. The system 600 may further include a control system 620, including a memory and a processor communicatively coupled thereto (e.g., realized by a computer system), for instrumental control and image acquisition. For example, the control system 620 may include functions to adjust laser intensity, parameters of imaging sensor, number of imaging cycles per session, selection of specific optical path for imaging, toggling between NIRS (or DOT) only or NIRS-MRI (or DOT-MRI) simultaneous imaging, as well as monitoring the status of various devices in the system 600.

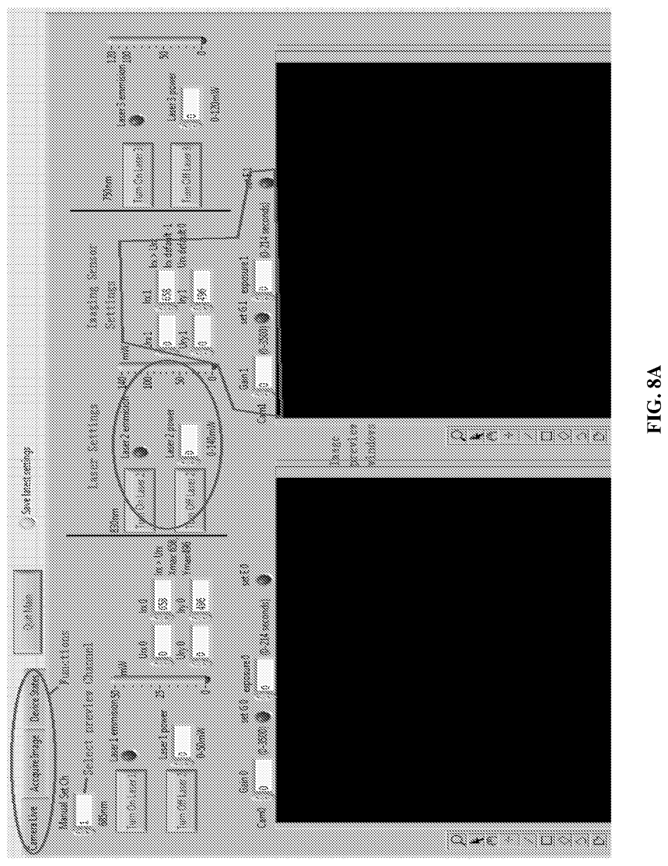

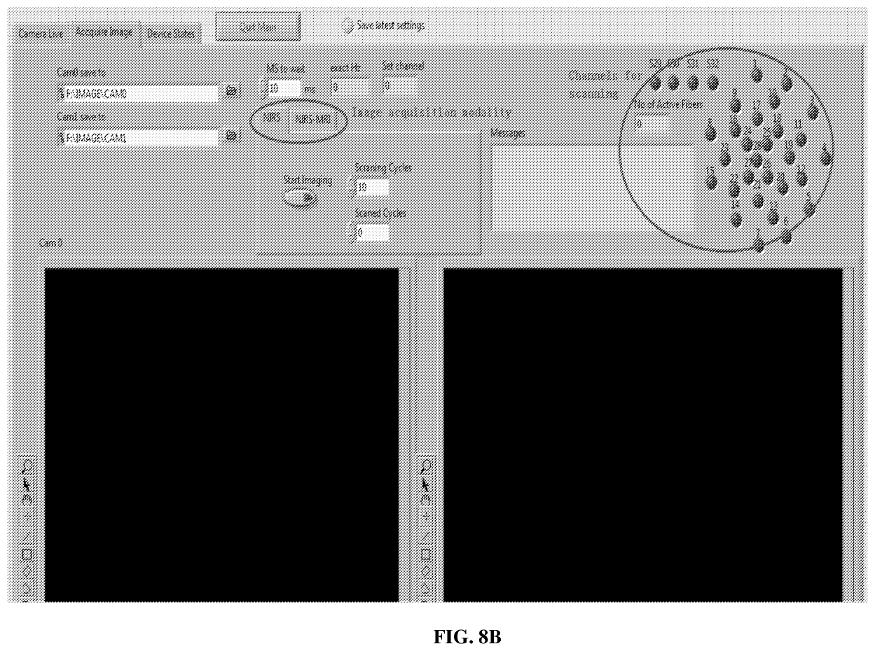

FIG. 7 depicts a flow chart showing various functions of the control system 620, and FIGS. 8A and 8B depict exemplary graphical user interface (GUI) associated with various functions of the control system for illustration purpose only and without limitation. For example, FIG. 8A depicts a GUI configured for a live preview of imaging, which allows users to adjust parameters for lasers and imaging sensors. FIG. 8B depicts a GUI configured for image acquisition, which allows user to select control modality and channels for scanning.

For better understanding, an example optical imaging technique will now be described according to various example embodiment of the present invention. Firstly, light from laser sources of different wavelengths are combined into a single optical path via a laser beam combiner, which is then fiber-coupled to an optical multiplexer that enables switching among multiple source fiber channels. Light is directed to and travels along one of the source channels and impinges onto the sample. Light attenuated from the tissue then travels along the detector channels, whereby the source channel and each detect channel form a single source-detector pair. In other words, each source channel and detector channel form a source-detector pair, probing a certain depth. For example, ‘M’ sources and ‘N’ detectors will form a total of M×N source-detector pairs. Light along the detector channels then split into two optical paths via a beamsplitter, whereby each optical path of light is detected by a corresponding CCD camera. The detected light intensity is encoded in raw CCD images, which may then be used for MATLAB post-processing (commonly known as image reconstruction) in order to compute optical properties (physiological information) of the sample.

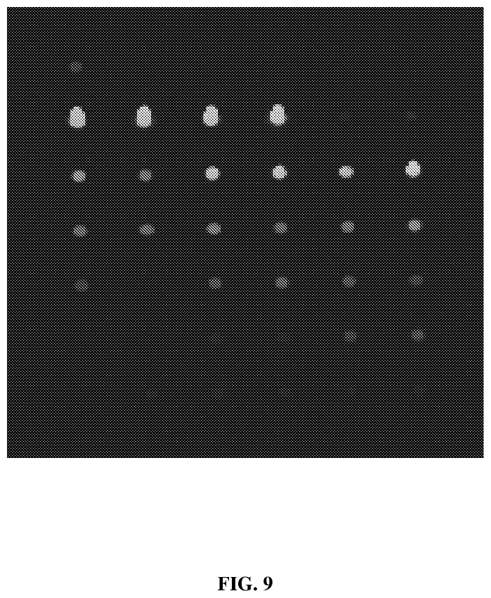

For illustration purpose only and without limitation, FIG. 9 depicts a raw CCD image corresponding to a particular source channel, where each bright spot corresponds to an individual detector channel, which forms a source-detector pair with the particular source channel.

An example optical imaging technique for DSCA will now be described according to various example embodiment of the present invention.

Suppose there are ‘M’ source channels and ‘N’ detector channels. The laser source may be directed to each source channel in succession (i.e., one by one), from source channel 1 to M. In this manner, one CCD image is generated for each source channel. After one imaging round, there will be M CCD images, one CCD image for each source channel. In various example embodiments, the above step may be repeated by a predetermined number of times, such as 50 times. As a result, for each source channel, there are 50 CCD images. Accordingly, the total number of images in this example may thus be 50×M.

As described above, in the example, for each source channel, there are 50 CCD images, and in each image, there are N detection spots. Therefore, for each source-detector pair (m, n) where m=1, 2, 3 . . . M, n=1, 2, 3 . . . N, there are 50 temporal intensity values. In an example implementation, the 50 intensity values may be used to calculate blood flow index for each source-detector pair based on the equation: BFI=(I/std)², where I is the mean value of the 50 temporal intensity at the center of the detector channel, and std is the standard deviation of these intensity values. Subsequently, the same 3D reconstruction method may be applied to the BFIs.

An example data registration technique will now be described according to various example embodiment of the present invention. For spatial image registration between MRI and optical data, during imaging, the fiducial channels are filled up (e.g., entirely) with glycerin, which is MRI-visible and does not evaporate much during the period of data acquisition. It was surprisingly found that these glycerin-filled fiducial channels show up clearly on the MRI anatomical image, which can be used to indirectly derive the MRI-invisible source and detector locations in the MRI space, since the multimodal MRI-DOT probe with the specific arrangement of the fiducial channels, source channels and detector channels is customized and thus known beforehand.

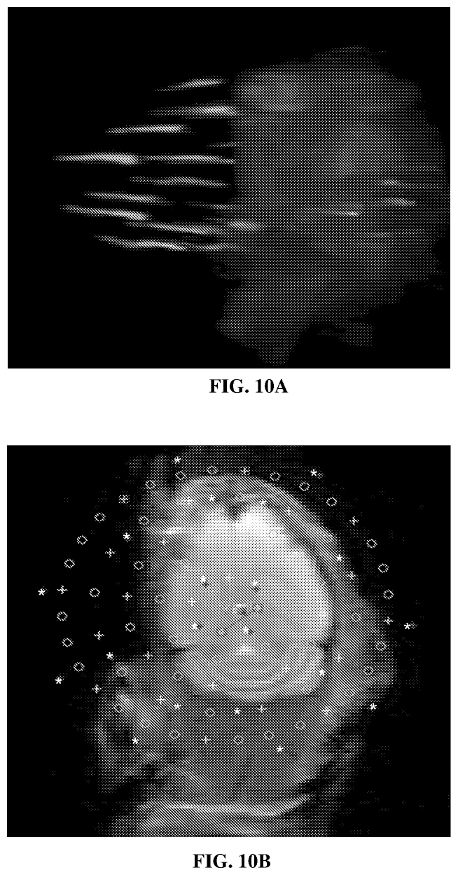

For illustration purpose only and without limitation, FIG. 10A depicts a MRI anatomical image of a mouse brain whereby the glycerin-filled fiducial channels on the probe show up as high intensity regions above the mouse brain. FIG. 10B depicts a MRI anatomical image whereby the MRI-invisible source channels (denoted by ‘+’ sign) and detector channels (denoted by a circle sign) locations derived from the glycerin-filled fiducial channels (denoted by a star sign) based on prior knowledge of the specific arrangement are overlaid on the MRI anatomical image.

In various example embodiments, combining MRI and optical techniques (e.g., DOT and DSCA) for simultaneous multi-modal data acquisition includes spatial registration (e.g., in terms of the fiducial markers in the probe design) and temporal registration (e.g., digital triggering by MRI).

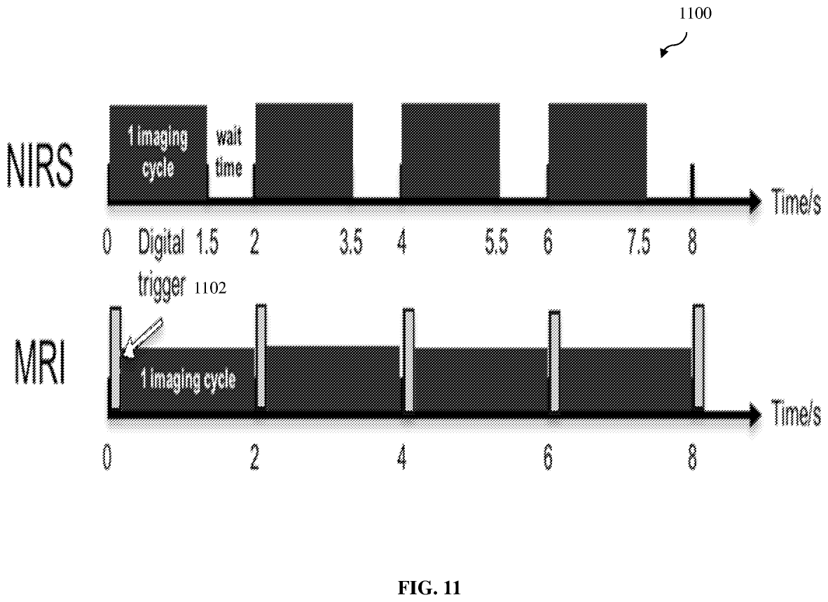

In relation to temporal registration, the NIRS (or DOT) data acquisition should be faster than the MRI data acquisition for each imaging cycle, so that the NIRS (or DOT) system may have sufficient time to wait for MRI digital triggering for the next imaging cycle of data acquisition. In various example embodiments, for temporal image registration, the MRI system may be configured to send a digital trigger (control signal) to the DOT system just before the start of the MRI data acquisition. This control signal triggers the DOT system to start acquiring optical imaging data at the same time for each imaging cycle of the multi-modal data acquisition. For illustration purpose only and without limitation, FIG. 11 depicts a timeline 1100 of the imaging cycles of the NIRS and MRI systems, and shows the digital trigger 1102 sent by the MRI system to start NIRS imaging for every imaging cycle Data acquisition imaging time for each imaging cycle may only be limited by the temporal resolution of both modalities, that is, how fast NIRS/MRI can acquire imaging data.

In various example embodiments, the intensity of each light spot on a CCD image acquired using a specific source channel, corresponds to the detected attenuated light for a specific source-detector pair. This acquired experimental data, together with information on source and detector coordinates (predetermined arrangement) and created mesh with nodes and elements, may be fed as input data into MATLAB for computing optical properties (absorption, scattering) based on a model on light propagation in tissues in a manner known in the art. For example, the optical properties may in turn be used to compute oxy- and deoxy-hemoglobin concentration, which in turn may be used to compute total haemoglobin concentration and oxygen saturation. For example, for data processing, various conventional techniques such as NIRFAST, a third-party MATLAB toolbox and NIRFAST-Slicer for ROI segmentation and mesh creation, developed by Dartmouth College may be utilized, such as described in M. Jermyn, H. Ghadyani, M. A. Mastanduno, W. Turner, S. C. Davis, H. Dehghani, and B. W. Pogue, “Fast segmentation and high-quality three-dimensional volume mesh creation from medical images for diffuse optical tomography,” J. Biomed. Opt. 18 (8), 086007 (Aug. 12, 2013) and “H. Dehghani, M. E. Eames, P. K. Yalavarthy, S. C. Davis, S. Srinivasan, C. M. Carpenter, B. W. Pogue, and K. D. Paulsen, “Near infrared optical tomography using NIRFAST: Algorithm for numerical model and image reconstruction,” Communications in Numerical Methods in Engineering, vol. 25, 711-732 (2009)”, the contents of which are hereby incorporated by reference in their entirety for all purposes.

In various example embodiments, the total number of images that are used or required for the blood flow calculation is a minimum of 15. In this regards, in various example embodiments, the number of source channels (M) may be determined based on the total number of images used or required, such as higher than the total number of images required. For example, if M is too small, the noise level of the optical imaging data may be high. On the other hand, if M is too large, it may take too much time to acquire the optical imaging data. Therefore, the number of source channels may be determined based on, but not limited to, such factors. For example, in the case of the total number of images required being 15, then the number of source channels may be set in the range of 15 to 100.

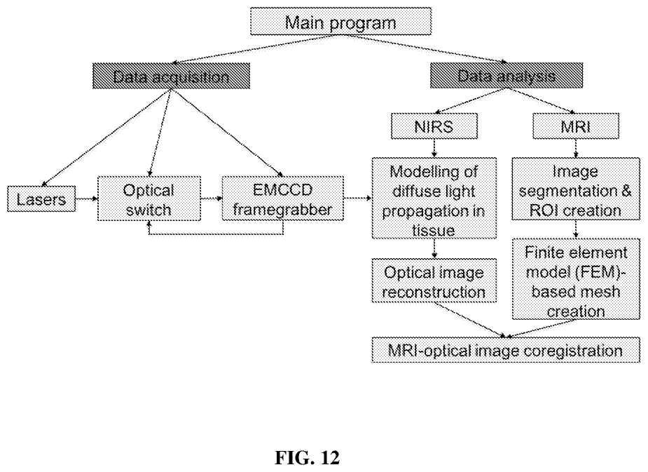

FIG. 12 depicts a flow diagram showing an overview of the method of multi-modal imaging of tissue as described herein according to various example embodiments.

In various example embodiments, for the DSCA, the number of source fiber channels (M) may be in the range of 2 to 80, and the number of detector fiber channels (N) may be in the range of 2 to 100. By way of an example, the exemplary probe 500 shown in FIG. 5 has 28 source fiber channels and 49 detector fiber channels.

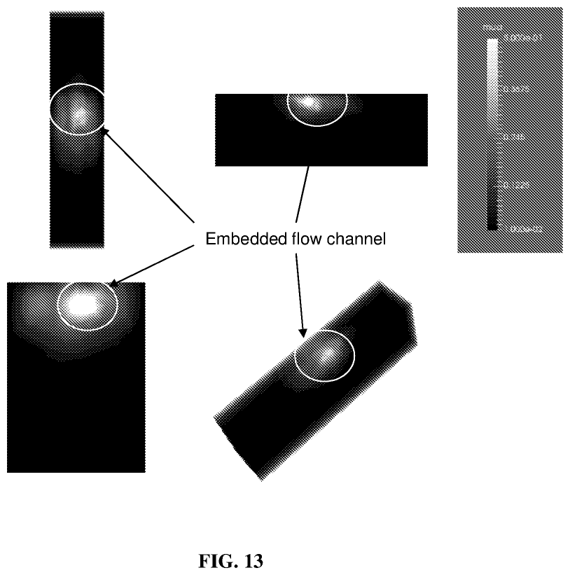

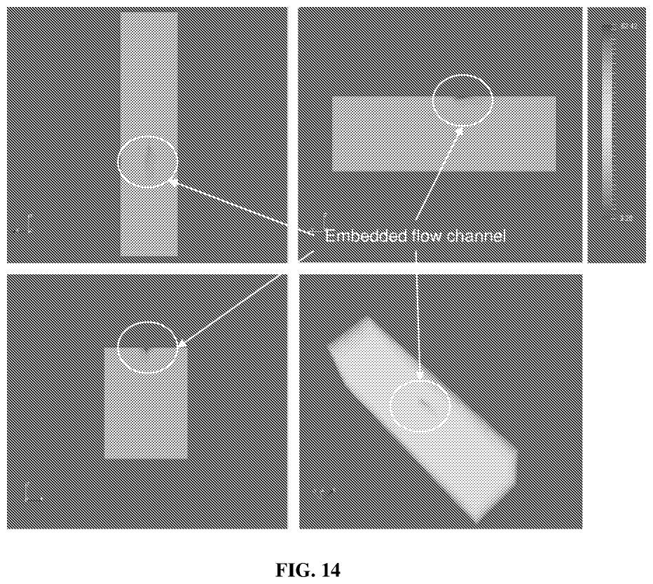

For illustration, diffuse optical measurements were performed on a phantom using the probe together with the multimodal imaging system according to an example embodiment. During data acquisition, the probe was placed on a flow phantom with a mixture of absorbing India ink and scattering Intralipid solution being pumped through a channel in the phantom at 2 ml/min using a syringe pump. Raw images were collected from the sensors and successfully used for image reconstruction in MATLB to yield 3D maps of absorption (FIG. 13) and flow (FIG. 14) within the phantom. This phantom study demonstrates that the probe can be used to collect optical imaging data, which in turn is used to provide information on oxygenation (function of absorption) and perfusion (function of flow), which can be correlated with MRI BOLD and perfusion measurements respectively.

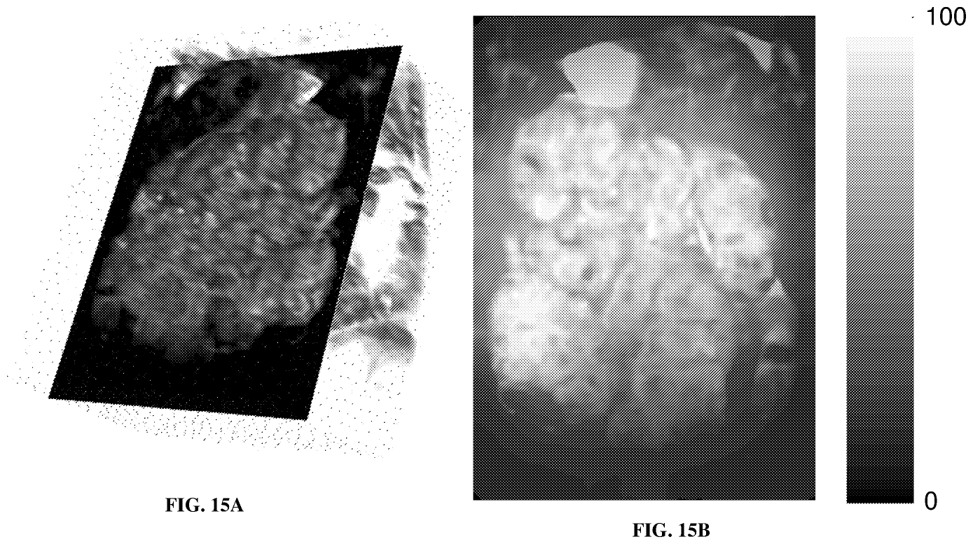

For illustration purpose and without limitation, FIGS. 15A and 15B depict images relating to overlaid optical data from NIRS (oxygen saturation) and MRI image stack. In particular, FIG. 15A shows a 3D segmented liver tumour from MRI DICOM stack along with a 2D MRI layer as shown in FIG. 15B with overlaid optical data. The 2D MRI layer image is overlaid by an optical saturation map (StO2) (varying between 0 and 100) calculated using NIRS source-detector geometry.

Accordingly, the method of multi-modal imaging described according to various embodiments possesses a number of advantages, such as but not limited to: (1) multimodal high-density optical imaging array coupled with high-field high-sensitivity MRI surface coil catered towards localized high-resolution imaging with high detection sensitivity and specificity; (2) source-detector arrangement can be customized and optimized for desired resolution-penetration depth configuration for various imaging applications; (3) MRI-visible fiducial markers specially designed for MRI-optical image coregistration; (4) combination of diffuse optical tomography and diffuse speckle contrast analysis as an integrated diffuse optical tomographic technique to detect tissue oxygenation and perfusion simultaneously; (5) validation of oxygen saturation and bloodflow data from optical imaging with fMRI blood-oxygen-level-dependent (BOLD) and blood perfusion data from MRI respectively; (6) computation of new physiological parameters from individual measurements, that is, oxygen saturation+bloodflow=metabolic rate of oxygen consumption; and (7) enable 3D images (3D reconstruction) of blood flow to be obtained (e.g., as a result of the multi-source/detector probe design and data analysis technique associated with the method), whereas conventional DSCA technique only allowed a point measurement of blood flow.

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

What is claimed is:

1. A method for multi-modal imaging of tissue, the method comprising:
providing a fiber probe over a target area of the tissue, the fiber probe comprising a plurality of source fiber channels, a plurality of detector fiber channels and a plurality of fiducial marker channels, the plurality of source fiber channels, the plurality of detector fiber channels and the plurality of fiducial marker channels having a predetermined spatial arrangement in the fiber probe;
directing light through each of the plurality of source fiber channels of the fiber probe to the target area in succession for performing optical imaging of the target area;
obtaining optical imaging data of the target area based on attenuated light from the target area detected through the plurality of detector fiber channels due to said light directed to the target area;
obtaining magnetic resonance imaging (MRI) data of the target area based on a MRI of the target area; and
coregistering the optical imaging data and the MRI data based on a plurality of fiducial features captured in the MRI data corresponding to the plurality of fiducial marker channels of the fiber probe.

2. The method according to claim 1, wherein the optical imaging data comprises at least one set of optical images, each set comprising a plurality of optical images, and each optical image generated based on said attenuated light detected through the plurality of detector fiber channels due to said light directed through a respective one of the plurality of source fiber channels.

3. The method according to claim 2, wherein each optical image comprises a plurality of light spots, each of the plurality of light spots corresponding to the attenuated light detected from a respective one of the plurality of detector fiber channels due to said light directed through the source fiber channel associated with the optical image, and
wherein the source fiber channel forms a source-detector pair with each respective one of the plurality of detector fiber channels, and each of the plurality of light spots is generated based on a respective one of the plurality of source-detector pairs.

4. The method according to claim 3, wherein the plurality of source-detector pairs is arranged in the fiber probe so as to have a range of source-detector distances.

5. The method according to claim 4, wherein the range of source-detector distances is from about 1 mm to about 60 mm.

6. The method according to claim 5, wherein each of the fiducial marker channels has disposed therein a material that is capable of being captured by MRI.

7. The method according to claim 3, wherein the MRI and the optical imaging of the target area are performed simultaneously, and said coregistering the optical imaging data and the MRI data comprises temporal coregistering and spatial coregistering the optical imaging data and the MRI data.

8. The method according to claim 7, further comprising, for an imaging cycle, sending a trigger signal from a MRI system configured to perform the MRI to an optical imaging system configured to perform the optical imaging to start the optical imaging for facilitating said temporal coregistering the optical imaging data and the MM data.

9. The method according to claim 7, wherein said spatial coregistering comprising spatially aligning a MRI image of the MRI data and an optical image of the optical imaging data based on positions of the plurality of fiducial features captured in the MRI image and positions of the plurality of light spots captured in the optical image, and based on the predetermined spatial arrangement of the plurality of detector fiber channels and the plurality of fiducial marker channels in the fiber probe.

10. The method according to claim 1, wherein said optical imaging is based on a diffuse optical imaging technique.

11. A system for multi-modal imaging of tissue, the system comprising:
an optical imaging system comprising:
a fiber probe configured to be provided over a target area of the tissue during the multi-model imaging of the target area, the fiber probe comprising a plurality of source fiber channels, a plurality of detector fiber channels and a plurality of fiducial marker channels, the plurality of source fiber channels, the plurality of detector fiber channels and the plurality of fiducial marker channels having a predetermined spatial arrangement in the fiber probe;
an optical switch configured to direct light through each of the plurality of source fiber channels of the fiber probe to the target area in succession for performing optical imaging of the target area; and
an image capturing module configured to obtain optical imaging data of the target area based on attenuated light from the target area detected through the plurality of detector fiber channels due to said light directed to the target area;
a magnetic resonance imaging (MRI) system comprising:
an image capturing module configured to obtain MRI data of the target area based on a MRI of the target area; and
an image coregistration module configured to coregister the optical imaging data and the MRI data based on a plurality of fiducial features captured in the MRI data corresponding to the plurality of fiducial marker channels of the fiber probe.

12. The system according to claim 11, wherein the optical imaging data comprises at least one set of optical images, each set comprising a plurality of optical images, and each optical image generated based on said attenuated light detected through the plurality of detector fiber channels due to said light directed through a respective one of the plurality of source fiber channels.

13. The system according to claim 12, wherein each optical image comprises a plurality of light spots, each of the plurality of light spots corresponding to the attenuated light detected from a respective one of the plurality of detector fiber channels due to said light directed through the source fiber channel associated with the optical image,
wherein the source fiber channel forms a source-detector pair with each respective one of the plurality of detector fiber channels, and each of the plurality of light spots is generated based on a respective one of the plurality of source-detector pairs.

14. The system according to claim 13, wherein the plurality of source-detector pairs is arranged in the fiber probe so as to have a range of source-detector distances.

15. The system according to claim 14, wherein the range of source-detector distances is from about 1 mm to about 60 mm.

16. The system according to claim 15, wherein each of the fiducial marker channels has disposed therein a material that is capable of being captured by the MRI.

17. The system according to claim 13, wherein the MRI and the optical imaging of the target area are performed simultaneously, and said coregistering the optical imaging data and the MRI data comprises temporal coregistering and spatial coregistering the optical imaging data and the MRI data.

18. The system according to claim 17, wherein the MRI system is configured to, for an imaging cycle, send a trigger signal to the optical imaging system to start the optical imaging of the target area for facilitating said temporal coregistering the optical imaging data and the MRI data.

19. The system according to claim 17, wherein said spatial coregistering comprising spatially aligning a MRI image of the MRI data and an optical image of the optical imaging data based on positions of the plurality of fiducial features captured in the MRI image and positions of the plurality of light spots captured in the optical image, and based on the predetermined spatial arrangement of the plurality of detector fiber channels and the plurality of fiducial marker channels in the fiber probe.

20. The system according to claim 11, wherein the optical imaging system is a diffuse optical imaging system.
